(19) United States
(12) Patent Application Publication
(10) Pub. No.: US 2024/0148691 A1
(43) Pub. Date: May 9, 2024

(54) Treatment of Hypertension-Related and Vascular Diseases

(71) Applicant: The Medical College of Wisconsin, Inc., Milwaukee, WI (US)

(72) Inventors: Andrey Sorokin, Whitefish Bay, WI (US); Doris Benbrook, Oklahoma City, OK (US)

(21) Appl. No.: 18/546,633

(22) PCT Filed: Feb. 23, 2022
(86) PCT No.: PCT/US2022/017513

§ 371 Date: Aug. 16, 2023

Related U.S. Application Data
(60) Provisional application No. 63/152,692, filed on Feb. 23, 2021.

(51) Int. Cl. A61K 31/382; A61K 45/06; A61P 9/12; A61P 13/12

(52) U.S. Cl. CPC A61K 31/382; A61K 45/06; A61P 9/12; A61P 13/12

(57) ABSTRACT

The present invention provides methods of reducing, inhibiting, preventing and treating vascular diseases. More specifically, the present disclosure provides methods of treating vascular diseases with the modulator of p66Shc signaling SHetA2.

### CROSS-REFERENCE TO RELATED APPLICATIONS

This application claims priority to U.S. Provisional Application No. 63/152,692 filed on Feb. 23, 2021, the contents of which is incorporated herein by reference in its entirety.

### STATEMENT REGARDING FEDERALLY SPONSORED RESEARCH

This invention was made with government support under grant number HL147976-0 awarded by the National Institutes of Health. The government has certain rights in this invention.

### BACKGROUND

Diabetes and hypertension can impair microvascular responsiveness and structural changes leading to oxidative stress, microvascular damage and kidney disease in the majority of patients. Microvascular reactivity of renal blood vessels is reduced in two major causes of end stage renal disease: hypertension-induced nephropathy (HN) and diabetic nephropathy. One of the highlights of vascular dysfunction in HN is the loss of autoregulation of blood resistance vessels, and resultant kidney damage caused by high blood pressure (hypertension). At this time, there are no known treatments which restore microvascular reactivity in hypertension-induced nephropathy. For treatment of hypertension in general, the anti-hypertensive drugs market is predicted to exceed USD 41,123.2 Million by 2023. Current pharmaceutical treatments for hypertension have significant side effects.

There is a need in the art for new methods to regulate microvascular reactivity and in turn treat diabetic nephropathy or hypertension and the resultant hypertension-induced nephropathy.

### SUMMARY

The present invention provides compositions and methods for treating hypertension related diseases and to reduce or inhibit hypertension-induced conditions including hypertension-induced nephropathy. The present invention demonstrates that the small molecule drug SHetA2 mitigates renal damage and restores microvascular responses in hypertensive and diabetic rats.

In one aspect of the current disclosure, methods of treating, reducing or inhibiting a hypertension-linked disease in a subject in need thereof are provided. In some embodiments, the methods comprise administering a therapeutically effective amount of SHetA2 or composition comprising SHetA2 to treat the hypertension-linked disease. In some embodiments, the blood pressure in the subject is not reduced. In some embodiments, the hypertension-linked disease comprises hypertension-induced nephropathy. In some embodiments, the method reduces urinary protein excretion in the subject. In some embodiments, the method improves autoregulation of blood vessels in the subject.

In another aspect of the current disclosure, methods of treating p66Shc-mediated vascular diseases in a subject in need thereof are provided. In some embodiments, the methods comprise administering a therapeutically effective amount of SHetA2 or a composition comprising SHetA2 to treat the vascular disease. In some embodiments, the vascular disease is a hypertension-induced disease. In some embodiments, the vascular disease is hypertension-induced nephropathy or diabetic nephropathy. In some embodiments, the method reduces urinary protein excretion in the subject. In some embodiments, the method improves autoregulation of blood vessels.

In another aspect of the current disclosure, methods of reducing or inhibiting hypertension-induced nephropathy are provided. In some embodiments, the methods comprise administering a therapeutically effective amount of SHetA2 or a composition comprising SHetA2 to a subject in need thereof to reduce or inhibit the hypertension-induced nephropathy. In some embodiments, the method reduces urinary protein excretion in the subject. In some embodiments, the method improves autoregulation of blood vessels.

In another aspect of current disclosure, methods of reducing or inhibiting high blood pressure related kidney damage in a subject in need thereof are provided. In some embodiments, the method comprises administering a therapeutically effective amount of SHetA2, or a composition comprising SHetA2 to a subject in need thereof to reduce or inhibit high blood pressure related kidney damage. In some embodiments, the method reduces urinary protein excretion in the subject. In some embodiments, the method improves autoregulation of blood vessels.

In another aspect of the current disclosure, kits are provided. In some embodiments, the kits comprise SHetA2 for treatment of vascular disease. In some embodiments, the kits comprise instructions to administer SHetA2 for treatment of diabetic nephropathy or hypertension-induced nephropathy. In some embodiments, the kits further comprise an additional drug that reduces blood pressure in a subject. In some embodiments, the drug comprises a diuretic, an angiotensin converting enzyme (ACE) inhibitor, an angiotensin II receptor blocker, a calcium channel blocker, an alpha blocker, a beta blocker, an alpha-beta blocker, an aldosterone agonist, a renin inhibitor, or a central acting agent.

### BRIEF DESCRIPTION OF THE DRAWINGS

FIG. 1 shows a cartoon diagram of the proposed mechanism of action and shows the compound SHetA2.

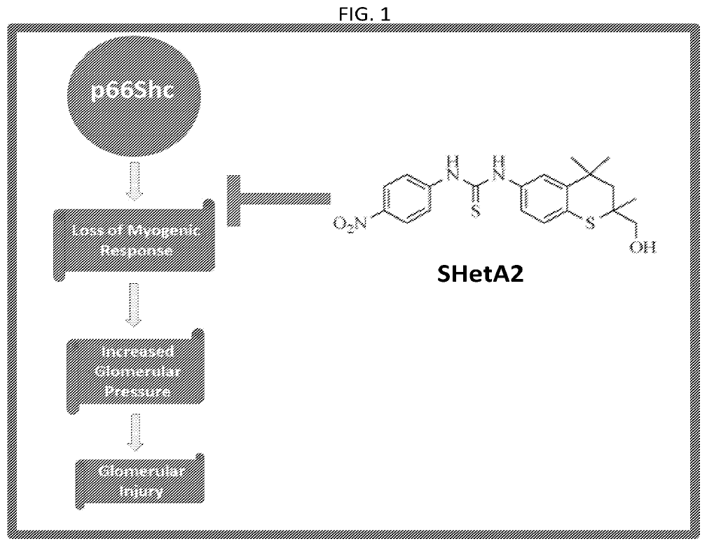

FIG. 2 shows a diagram of causes of End Stage Renal Disease.

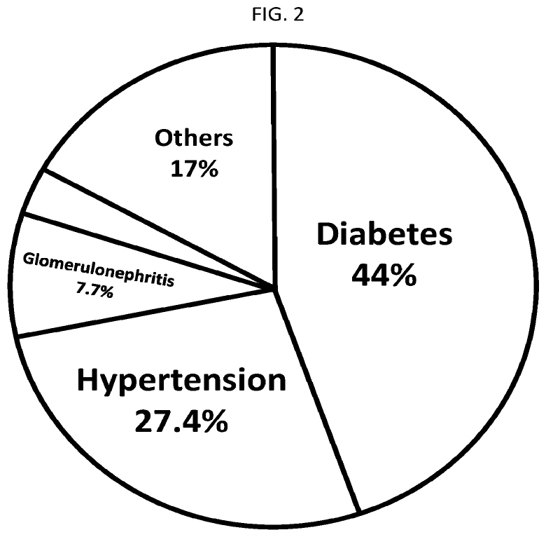

FIG. 3 shows expression of adaptor protein p66Shc (Blue stain: target of SHetA2) in afferent arterioles of hypertensive rats and resulting loss of myogenic response leading to glomerular damage in hypertension-induced nephropathy.

FIG. 4 shows is a picture of p66Shc expression in smooth muscle cells of renal afferent arterioles (blue stain).

FIG. 5 shows p66Shc knockout restored renal microvascular responses in hypertensive rats. Microvascular responses of renal afferent preglomerular arterioles to perfused pressure. Juxtamedullary nephron vasculature was isolated, and an afferent arteriole was monitored continuously by videomicroscopy. SS/BN2 refers to BN chromosome replacement into SS background. Thus, SS/BN2 are not expected to be salt-sensitive and represent a control for the SS strain.

FIG. 6 demonstrates SHetA2 partially restored renal microvascular responses in renal afferent preglomerular arterioles in vitro.

FIG. 7 is a diagram of the experimental design of chronic SHetA2 treatment of WT and p66Shc-KO rates.

FIG. 8 demonstrates SHetA2 restored renal microvascular responses in hypertensive rats.

FIG. 9 demonstrates SHetA2 restored renal microvascular responses in hypertensive rats.

FIG. 10 explains how expression of p66Shc in smooth muscle cells of afferent arterioles causes renal microvascular dysfunction.

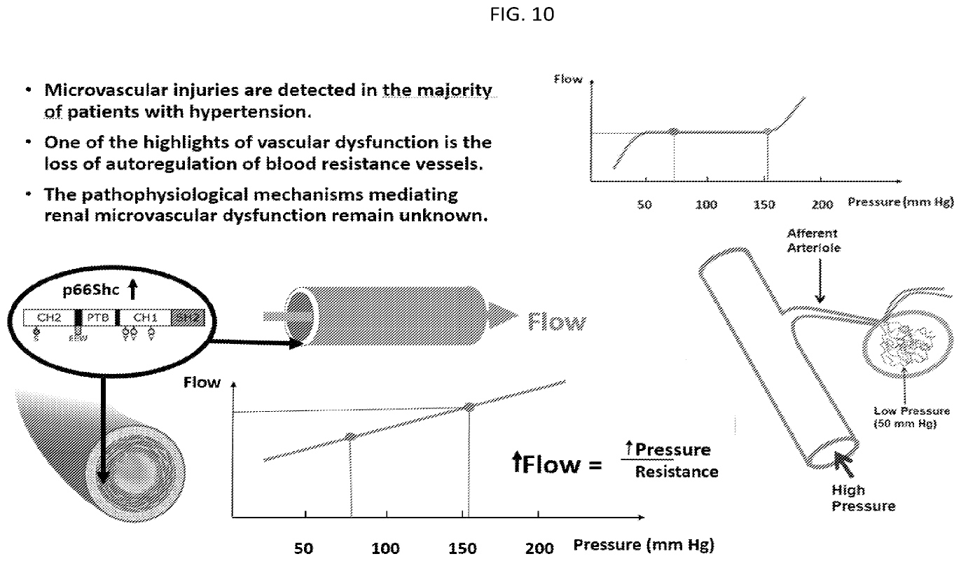

FIG. 11 shows a schematic of p66Shc translocation to mitochondria. p66Shc, upon Ser36 phosphorylation, in wild type control SS rats is translocated to the mitochondria, where it contributes to production of ROS. p66Shc is absent in p66Shc-KO rats. In p66Shc-S36A rats, single amino acid substitution of Ser36 to Ala prevents mitochondrial translocation and retains p66Shc in cytoplasm. In p66Shc-Del rats the inventors detected enhanced ROS production which is likely due to predominant localization of p66Shc-Del in mitochondria.

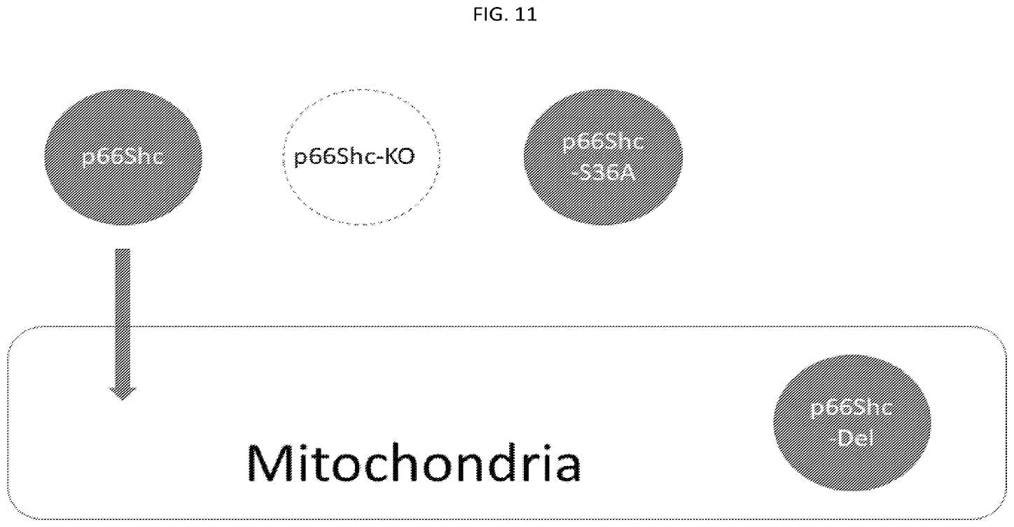

FIG. 12 shows a schematic of hypothetical role for p66Shc in hypertension-induced nephropathy and diabetic nephropathy.

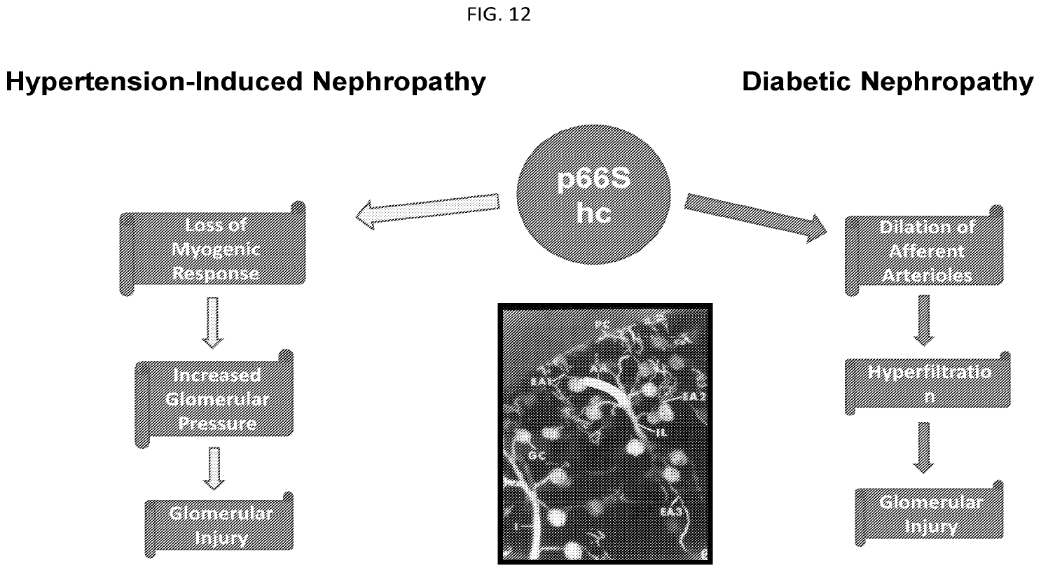

FIG. 13 shows a schematic demonstrating a hypothetical mechanism for SHetA2 in regulating microvascular reactivity of renal blood vessels.

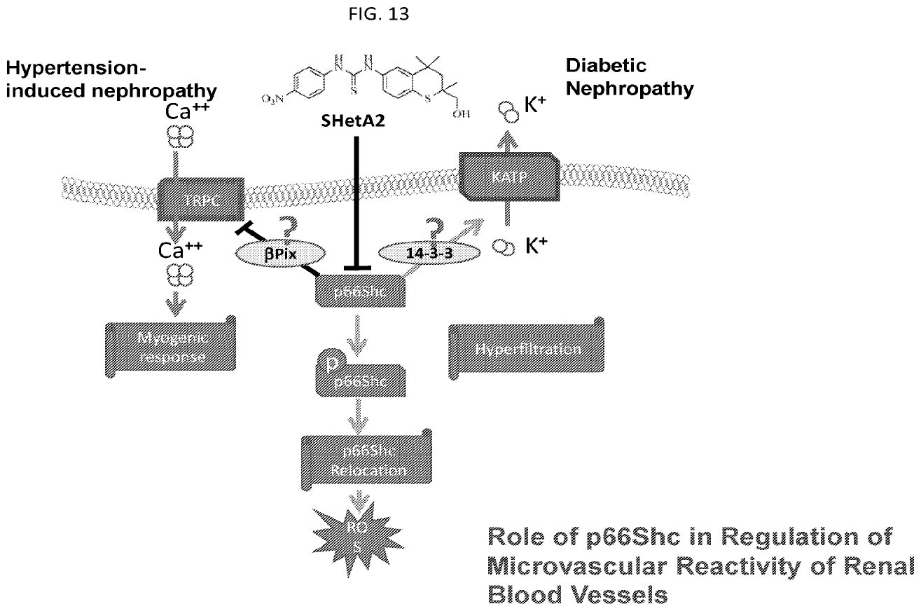

FIG. 14 shows dose-dependent decrease in vessel diameter in response to SHetA2 treatment.

FIG. 15 shows a schematic representation of study design for FIG. 18. Rats were weaned at 3 weeks of age and transferred from 0.4% (Teklad 7034) to 1% salt diet (Purina 5001) for an additional 3 weeks before treatment. Kolliphor® HS 15 is a potent non-ionic solubilizer and emulsifying agent, with low toxicity. Ideal for solubilizing low-solubility compounds in microemulsions.

FIG. 16 demonstrates the lack of effect of long-term treatment of animals with SHetA2 upon body weight and increase of blood pressure.

FIG. 17 demonstrates a decrease in vessel diameter in response to SHetA2 treatment in both male and female rats subsequent to chronic administration of SHetA2.

FIG. 18 demonstrates that SHetA2 treatment restores renal microvascular responses in hypertensive rats.

FIG. 19 demonstrates that SHet2A administration reduces or prevents urinary protein excretion in wild type and S36A salt-sensitive animals, but not in p66ShcKO rats. These data indicate that the effect of SHetA2 is linked to p66Shc signaling.

FIG. 20 demonstrates that SHetA2 reduces or prevents development of signs of diabetic nephropathy (dilation of afferent arterioles) in rats treated with streptozotocin (STZ) to induce diabetes. The absence of effect in p66ShcKO rats indicates that the effect of SHetA2 is linked to p66Shc signaling.

FIG. 21 demonstrates that SHetA2 reduces or prevents urinary protein excretion in diabetic animals and reduces signs of diabetic nephropathy in the same.

FIG. 22 shows the action of glibenclamide and demonstrates that glibenclamide is effective in attenuating sings of diabetic nephropathy in diabetic rats, but not diabetic p66ShcKO rats.

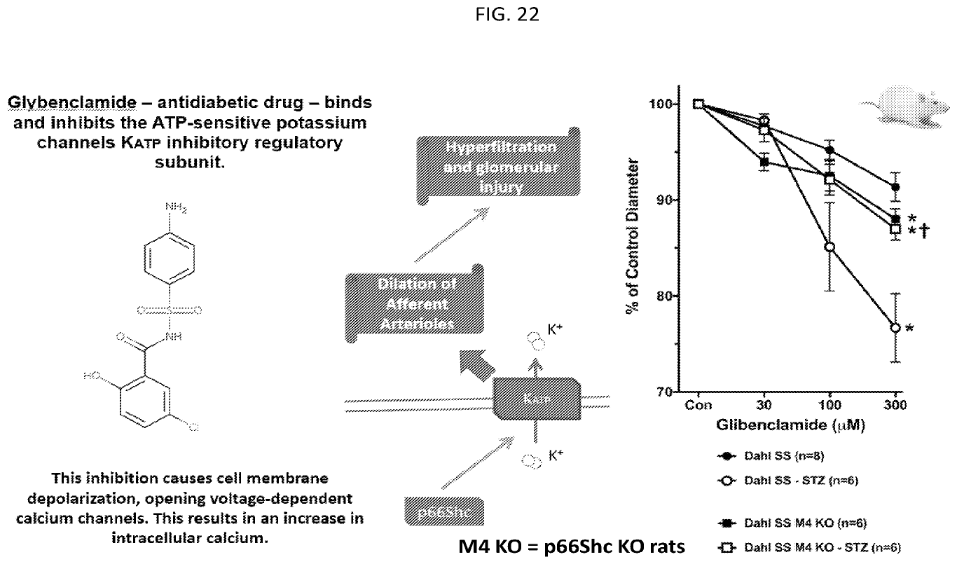

FIG. 23 demonstrates that the lack of glibenclamide effect in diabetic rats treated with SHetA2, which supports the hypothesis that SHetA2 acts by mitigating p66Shc-induced activation of K_{ATP} channels.

### DETAILED DESCRIPTION

Diabetes and hypertension can impair microvascular responsiveness and structural changes leading to oxidative stress, microvascular damage and kidney disease in the majority of patients.

This disclosure describes a new therapeutic strategy to prevent and treat these diseases by controlling microvascular reactivity of renal blood vessels. The strategy targets the Src homology 2 domain-containing adaptor protein (p66Shc), which mediates cellular sensitivity to oxidative stress p66Shc regulates renal vascular tone in isolated preglomerular arterioles and promotes renal vascular dysfunction. The mechanism of action of p66Shc is linked to inhibition of transient receptor potential canonical (TRPC) channel activity, which limits the increase in intracellular Ca²⁺-induced activation of calcium dependent intracellular signaling pathways and impairs vascular responses. Excessive signaling of p66Shc has been linked to age-related pathologies. Thus, excessive p66Shc activity is implicated in the loss of microvascular reactivity during the progression of both hypertension-induced nephropathy and diabetic nephropathy.

Microvascular reactivity of renal blood vessels is reduced in two major causes of end stage renal disease: hypertension-induced nephropathy (HN) and diabetic nephropathy. One of the highlights of vascular dysfunction in HN is the loss of autoregulation of blood resistance vessels. Using rat model of salt-sensitive (SS) HN we have previously shown that overexpression of adaptor protein p66Shc is implicated in the loss of microvascular reactivity during the progression of HN. The inventors discovered the ability of a flexible heteroarotinoid called Sulfur Heteroarotinoid A2 (SHetA2, pronounced Ess-Het-Aye-two), known to release p66Shc from binding to a chaperone protein called mortalin, to restore renal microvascular responses in hypertensive rats, and thus have the ability to treat hypertension-induced nephropathy.

Two independently generated and genetically distinct p66Shc-KO rat mutants demonstrated similar restoration of renal microvascular responses. Mortalin assures proper folding and function of proteins and protein complexes. SHetA2 disruption of mortalin and other chaperone/client protein complexes, causes degradation, intracellular relocalization, and/or gain-of-function or loss-of-function of the client protein, depending on the specific client protein and cellular context. The effects of SHetA2 disruption of the mortalin/p66Shc complex at the molecular and cellular levels are unknown. The inventors have shown that disruption of mortalin binding to its client proteins can cause re-localization of the client protein within the cell and/or degradation of the protein. It was not obvious if SHetA2 treatment would reduce or increase kidney microvascular damage, because SHetA2 disruption of mortalin/p66Shc complexes could result in either increase or decrease in levels or activity of p66Shc. In some instances, mortalin has been shown to inhibit the activities of its client proteins, while in other cases mortalin supports activity of its client proteins. However, using genetically modified Dahl salt sensitive (SS) rats we demonstrate herein that SHetA2 can prevent salt-induced microvascular damage in the kidney. The present disclosure also demonstrates the restoration of renal vascular function as evaluated by studying microvascular responses to purinergic activation and perfused pressure in the control SS rats and SS rats treated with compound SHetA2.

As used herein, “SHetA2” refers to a compound having the formula:

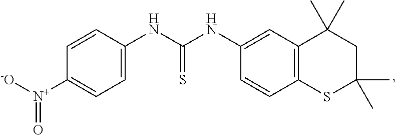

or a pharmaceutically acceptable salt thereof.

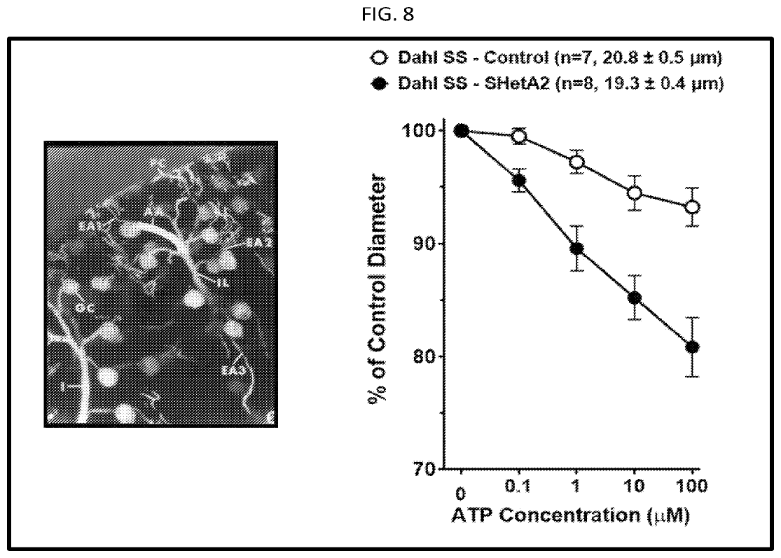

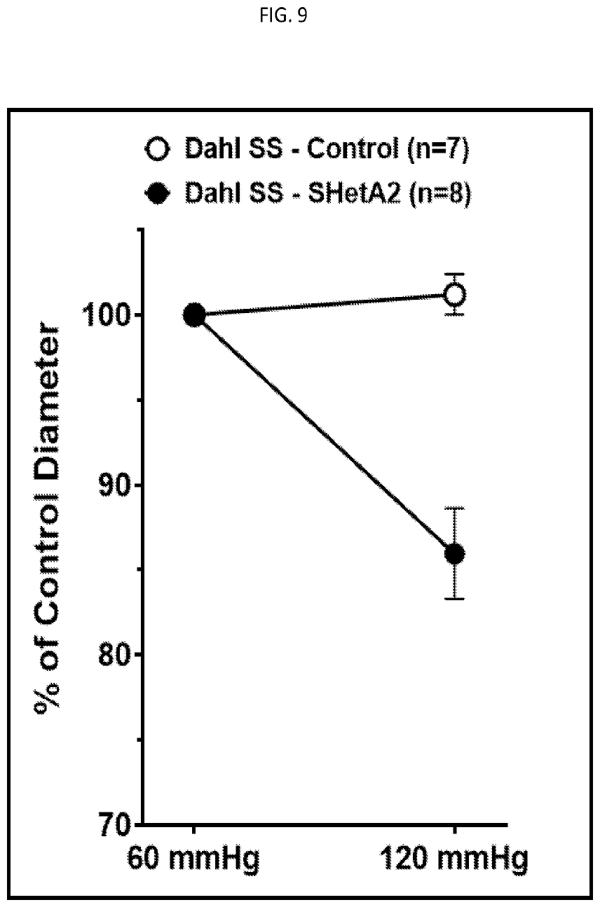

As demonstrated in the examples and Exhibit A, at the physiological level, SHetA2 treatment of SS HN rats resulted in statistically significant decrease of urinary protein excretion when compared to control-treated rats, indicating that SHetA2 preserves kidney function in hypertensive rats. Vascular reactivity of renal afferent arterioles was measured using the juxtamedullary nephron technique approximately 24 hours after final treatment (18 weeks) (FIG. 9). Remarkably, SHetA2 restored renal microvascular responses in hypertensive rats, as detected by response of afferent arterioles either to increasing perfusion pressure or to ATP, known mediator of afferent arteriolar autoregulatory responses (FIG. 8). Since compound SHetA2 demonstrates reasonable pharmacokinetics and lack of mutagenicity, carcinogenicity and toxicity, it can be used for therapeutic intervention of p66Shc-mediated vascular diseases, namely hypertension-induced nephropathy and diabetic nephropathy.

SHetA2 has the potential for preventing, reducing, and treating many complications of hypertension, including damage to the kidney, heart, eyes, thyroid, arteries, sleep and sexual function, and increase risk of stroke. Common hypertension-linked diseases or outcomes are known and include, but are not limited to, heart attack, high blood pressure, which can damage arteries that can become blocked and prevent blood flow to the heart muscle; stroke, high blood pressure can cause blood vessels in the brain to clog more easily or even burst; heart failure, the increased workload from high blood pressure can cause the heart to enlarge and fail to supply blood to the body; kidney disease or failure, high blood pressure can damage the arteries around the kidneys and interfere with their ability to filter blood effectively; vision loss, high blood pressure can strain or damage blood vessels in the eyes; sexual dysfunction, high blood pressure can lead to erectile dysfunction in men or lower libido in women; angina, over time, high blood pressure can lead to heart disease or microvascular disease (MVD), where angina, or chest pain, is a common symptom; peripheral artery disease (PAD), atherosclerosis caused by high blood pressure can cause a narrowing of arteries in the legs, arms, stomach and head, causing pain or fatigue, among others. SHetA2 has the potential to be less toxic than current treatments for hypertension and hypertension related diseases. SHetA2 also works through a novel mechanism of action and therefore has the potential to be more effective than the other treatments. These current treatments are not sufficient, because nearly half a million deaths were caused by hypertension in 2018 in the US, despite use of these medications. SHetA2 has the potential to replace or complement these other treatments.

Use of SHetA2 for treatment of hypertension has the potential to be less toxic or possibly have no toxicity at all. SHetA2 works through a different mechanism than all of the current treatments described below and therefore is not likely to induce the same side effects. In extensive preclinical studies conducted by the NCI, oral SHetA2 was found to have no toxicity at doses 25-fold above the doses used in the rat hypertensive model. Animal models also showed that SHetA2 does not cause teratogenicity (birth defects) and does not have any detectable effect on heart function after months of treatment.

The present SHetA2 for treatment may prevent and, reduce or eliminate one or more side effects associated with the current hypertension drugs on the market. Medications used to treat high blood pressure include:

### Diuretics

Diuretics, sometimes called water pills, are medications that help your kidneys eliminate sodium and water from the body. These drugs are often the first medications tried to treat high blood pressure. There are different classes of diuretics, including thiazide, loop and potassium sparing. Which one a physician recommends depends on a subject's blood pressure measurements and other health conditions, such a kidney disease or heart failure. Diuretics commonly used to treat blood pressure include chlorthalidone, hydrochlorothiazide (Microzide) and others. A common side effect of diuretics is increased urination, which could reduce potassium levels. Subjects with a low potassium level, may benefit from adding a potassium-sparing diuretic—such as triamterene (Dyazide, Maxide) or spironolactone (Aldactone)—to the treatment.

### Angiotensin-Converting Enzyme (ACE) Inhibitors

These medications—such as lisinopril (Prinivil, Zestril), benazepril (Lotensin), captopril and others—help relax blood vessels by blocking the formation of a natural chemical that narrows blood vessels.

### Angiotensin II Receptor Blockers (ARBs)

These medications relax blood vessels by blocking the action, not the formation, of a natural chemical that narrows blood vessels. ARBs include candesartan (Atacand), losartan (Cozaar) and others.

### Calcium Channel Blockers

These medications—including amlodipine (Norvasc), diltiazem (Cardizem, Tiazac, others) and others—help relax the muscles of a subject's blood vessels, while some slow a subject's heart rate. Calcium channel blockers may work better for older people and people of African heritage than do ACE inhibitors alone.

### Additional Medications Sometimes Used to Treat High Blood Pressure

In cases where the blood pressure goal cannot be achieved with combinations of the above medications, the following may be prescribed:

### Alpha Blockers

These medications reduce nerve signals to blood vessels, lowering the effects of natural chemicals that narrow blood vessels. Alpha blockers include doxazosin (Cardura), prazosin (Minipress) and others.

### Alpha-Beta Blockers

Alpha-beta blockers block nerve signals to blood vessels and slow the heartbeat to reduce the amount of blood that must be pumped through the vessels. Alpha-beta blockers include carvedilol (Coreg) and labetalol (Trandate).

### Beta Blockers

These medications reduce the workload on a subject's heart and widen blood vessels, causing the heart to beat slower and with less force. Beta blockers include acebutolol, atenolol (Tenormin) and others. Beta blockers aren't usually recommended as the only medication that's prescribed, but they may be effective when combined with other blood pressure medications.

### Aldosterone Antagonists

These drugs also are considered diuretics. Examples are spironolactone and eplerenone (Inspra). These drugs block the effect of a natural chemical that can lead to salt and fluid buildup, which can contribute to high blood pressure. They may be used to treat resistant hypertension.

### Renin Inhibitors

Aliskiren (Tekturna) slows the production of renin, an enzyme produced by the kidneys that starts a chain of chemical steps that increases blood pressure.

### Vasodilators

These medications include hydralazine and minoxidil. They work directly on the muscles in the walls of a subject's arteries, preventing the muscles from tightening and arteries from narrowing.

### Central Acting Agents

These medications prevent the brain from telling the nervous system to increase heart rate and narrow blood vessels. Examples include clonidine (Catapres, Kapvay), guanfacine (Intuniv) and methyldopa

The present disclosure provides a method of treating hypertension in a subject, the method comprising administering a therapeutically effective amount of SHetA2 or composition comprising SHetA2 to treat the hypertension.

For purposes of the present invention, by “treating”, “treat”, or “treatment” the term describes the management of care for the purpose of combating the disease, condition or disorder, for example, to reduce or inhibit one or more symptom or condition associated with the disease, alleviating the symptoms or complications, or eliminating the disease, condition, or disorder. Specifically, for example, in one aspect, methods of treating a hypertension-linked disease in a subject in need thereof are provided. As used herein, “hypertension-linked disease” refers to any symptom, sign, syndrome, or disease that is caused, at least in part, by hypertension. Exemplary hypertension-linked diseases comprise hypertension-induced nephropathy, stroke, heart attack, etc.

As used herein, “therapeutically effective amount” refers to the amount or dose of the compound, upon single or multiple dose administration to the subject, which provides the desired effect in the subject under diagnosis or treatment.

A therapeutically effective amount can be readily determined by the attending diagnostician, as one skilled in the art, by the use of known techniques and by observing results obtained under analogous circumstances. In determining the effective amount or dose of compound administered, a number of factors can be considered by the attending diagnostician, such as: the species of the subject; its size, age, and general health; the degree of involvement or the severity of the disease or disorder involved; the response of the individual subject; the particular compound administered; the mode of administration; the bioavailability characteristics of the preparation administered; the dose regimen selected; the use of concomitant medication; and other relevant circumstances.

In some embodiments, the disclosed methods prevent or reduce a sign or symptom of hypertension, or a complication of hypertension. As used herein, “prevent” refers to delaying the onset of, reducing the incidence of, or reducing the severity of a sign or symptom, e.g., a sign or symptom of hypertension or a hypertension-linked disease, in a subject in need thereof (e.g., a subject displaying one or more symptoms associated with hypertension or complications of hypertension). In some embodiments, the disclosed methods reduce, inhibit, or prevent complications of hypertension, including damage to the kidney, heart, eyes, thyroid, arteries, sleep and sexual function, and stroke. As such, the methods disclosed herein encompass both therapeutic and prophylactic administration, especially in patients having or displaying one or more symptoms of associated with hypertension or complications of hypertension.

As used herein, “a subject in need thereof” may refer to a subject suffering from a hypertension-linked disease, or a subject having hypertension that may have or may develop one or more complications associated with hypertension. In some embodiments, a subject in need thereof refers to a subject suffering from a p66Shc-mediated vascular disease, e.g., hypertension-induced nephropathy or diabetic nephropathy. In some embodiments, a subject in need thereof refers to a subject in need of a reduction or inhibition of high blood pressure associated damage. In some embodiments, a subject in need thereof is a subject at risk of developing a sign or symptom of hypertension or a hypertension-related disease or complication. In some embodiments, a subject in need thereof refers to a subject at risk of developing a p66Shc-mediated vascular disease, e.g., hypertension-induced nephropathy or diabetic nephropathy.

In some embodiments, the methods of treating a hypertension-linked disease comprise administering a therapeutically effective amount of SHetA2, or a composition comprising SHetA2, to a subject in need thereof to treat the hypertension-linked disease, or reduce or inhibit microvascular disease consequences of hypertension. A composition comprising SHetA2, in some embodiments, refers to a pharmaceutical composition comprising SHetA2 and a pharmaceutically acceptable carrier. In some embodiments, the composition comprising SHetA2 may further comprise a drug that has a separate pharmacological mechanism of action to reduce or inhibit hypertension itself, or a hypertension-linked disease.

In some embodiments, the methods of preventing or reducing a hypertension-linked disease comprise administering a therapeutically effective amount of SHetA2, or a composition comprising SHetA2, to a subject in need thereof to treat the hypertension-linked disease, or prevent microvascular disease consequences of hypertension. A composition comprising SHetA2, in some embodiments, refers to a pharmaceutical composition comprising SHetA2 and a pharmaceutically acceptable carrier. In some embodiments, the composition comprising SHetA2 may further comprise an additional drug that has a separate pharmacological mechanism of action to reduce or inhibit hypertension itself, or a hypertension-linked disease.

In another aspect of the current disclosure, the treatment by administration of SHetA2, or a composition comprising SHetA2, results in an increase in the renal microvascular response within the subject. Renal blood flow (RBF) autoregulation, or simply “autoregulation of blood vessels” is a vital homeostatic mechanism that protects the kidney from elevations in arterial pressure that would be transmitted to the glomerular capillaries and cause injury. It also allows the kidney to maintain a relatively constant blood flow and glomerular filtration rate (GFR) necessary for the clearance of metabolic wastes while maintaining efficient recovery of filtered electrolytes and nutrients by the renal tubules. Two mechanisms contribute to autoregulation of RBF. The first is the myogenic response of preglomerular arterioles. Elevations in transmural pressure induce contraction of preglomerular arterioles, predominantly at the level of afferent arterioles. The other mechanism is tubuloglomerular feedback which acts in concert with the myogenic response. It senses changes in the concentration of sodium chloride in the tubular fluid reaching the macula densa cells in the distal tubule and adjusts the diameter of the afferent arteriole accordingly. Tubuloglomerular feedback serves as an effective autoregulatory mechanism because the sodium chloride concentration of the fluid reaching the macula densa is dependent on flow rate, which in turn, is related to the GFR and glomerular capillary pressure. Elevations in vascular resistance, especially in the renal circulation, are characteristic of hypertension. There is also generalized endothelial dysfunction associated with diminished vasodilatory responses to shear stress and other stimuli. In summary, diabetes and hypertension are associated with impairments in the autoregulation of renal blood flow. Thus, in some embodiments, the methods improve autoregulation of blood vessels in the subject. In some embodiments, the blood vessels are renal blood vessels. In some embodiments, the blood vessels are renal afferent preglomerular arterioles.

A key sign of kidney disease is an increase in protein excretion in the urine. The presence of albuminuria or proteinuria constitutes a sign of kidney damage and, together with the estimation of glomerular filtration rate, is based on the evaluation of chronic kidney disease. Proteinuria is a strong marker for progression of chronic kidney disease, and it is also a marker of increased cardiovascular morbidity or mortality. Filtration of albumin by the glomerulus is followed by tubular reabsorption, and thus, the resulting albuminuria reflects the combined contribution of these 2 processes. Dysfunction of both processes may result in increased excretion of albumin, and both glomerular injury and tubular impairment have been involved in the initial events leading to proteinuria. Thus, in some embodiments, the methods reduce urinary protein excretion in the subject.

The present disclosure further provides a method of preventing or treating p66Shc-mediated vascular diseases, the method comprising administering a therapeutically effective amount of SHetA2 or a composition comprising SHetA2 in order to prevent or treat the vascular disease. In some embodiments, the vascular disease is a hypertension-induced disease, for example, hypertension-induced nephropathy. In some embodiments, the vascular disease is diabetic nephropathy. In some embodiments, the method reduces urinary protein excretion in the subject. In some embodiments, the methods improve autoregulation of blood vessels in the subject. In some embodiments, the blood vessels are renal blood vessels. In some embodiments, the blood vessels are renal afferent preglomerular arterioles.

The present disclosure further provides methods of preventing, reducing or inhibiting hypertension-induced nephropathy, comprising administering a therapeutically effective amount of SHetA2 or a composition comprising SHetA2 to prevent or treat the hypertension-induced nephropathy. In some embodiments, the method reduces urinary protein excretion in the subject. In some embodiments, the methods improve autoregulation of blood vessels in the subject. In some embodiments, the blood vessels are renal blood vessels. As used herein “renal blood vessels” refers to any of the vasculature inside a kidney. In some embodiments, the blood vessels are renal afferent preglomerular arterioles.

In another aspect of the current disclosure, methods of reducing or inhibiting high blood pressure related damage (e.g., kidney damage) in a subject in need thereof are provided. In some embodiments, the methods comprise administering a therapeutically effective amount of SHetA2, or a composition comprising SHetA2 to a subject in need thereof to prevent, reduce, or inhibit high blood pressure related damage. As used herein “inhibiting high blood pressure related damage” refers to the ability of SHetA2, in some embodiments, to reduce signs and symptoms associated with high blood pressure, i.e., hypertension, without reducing the level of blood pressure itself. In some embodiments, the method reduces urinary protein excretion in the subject. In some embodiments, the methods improve autoregulation of blood vessels in the subject. In some embodiments, the blood vessels are renal blood vessels. In some embodiments, the blood vessels are renal afferent preglomerular arterioles.

In another aspect of the current disclosure, kits are provided. In some embodiments, the kits comprise SHetA2 for prevention or treatment of vascular disease. In some embodiments, the kits further comprise instructions to administer SHetA2 for treatment of diabetic nephropathy or hypertension-induced nephropathy. In some embodiments, the kits comprise a drug, i.e., a drug in addition to SHetA2, that reduces blood pressure in a subject. In some embodiments, the drug comprises a diuretic, an angiotensin converting enzyme (ACE) inhibitor, an angiotensin II receptor blocker, a calcium channel blocker, an alpha blocker, a beta blocker, an alpha-beta blocker, a renin inhibitor, or a central acting agent.

The SHetA2 used in the methods described herein is described in may be suitably formulated in a composition for administration to a subject. The present disclosure also provides compositions comprising SHetA2 and a pharmaceutically acceptable carrier. The compositions can be prepared in unit dosaged forms for administration to a subject. The amount and timing of administration are at the discretion of the treating clinician to achieve the desired outcome.

As used herein, “pharmaceutical composition” means therapeutically effective amounts of the SHetA2 together with a pharmaceutically acceptable carrier. “Pharmaceutically acceptable” carriers are known in the art and include, but are not limited to, for example, suitable diluents, preservatives, solubilizers, emulsifiers, liposomes, nanoparticles and adjuvants. Pharmaceutically acceptable carriers are well known to those skilled in the art.

The term subject refers to a mammal, preferably a human. In some embodiments, the subject is a human with hypertension. In some embodiments, the subject is a human with a p66Shc-mediated vascular disease.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-peritoneal administration, intra-arterial administration, intramuscular administration, intradermal administration, intrathecal administration, subcutaneous administration and administration through an intra-uterine device. Administration can be continuous or intermittent. In various aspects, a preparation can be administered therapeutically; that is, administered to treat an existing disease or condition.

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

The following examples are meant only to be illustrative and are not meant as limitations on the scope of the invention or of the appended claims.

### EXAMPLES

In the following example, the inventors describe the ability of SHetA2 to restore renal microvascular responses in hypertension-induced nephropathy

### Example 1

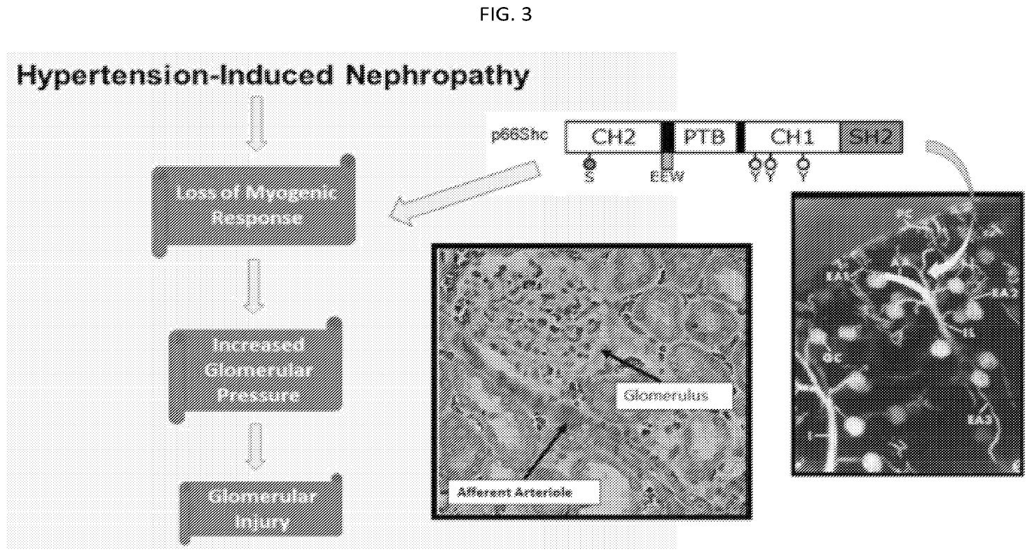

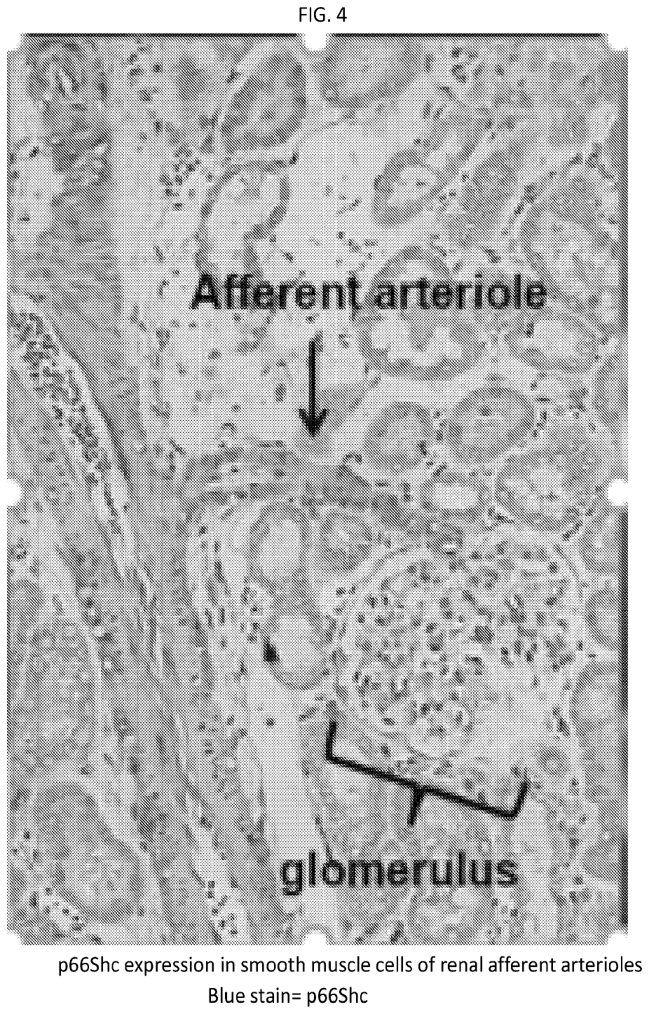

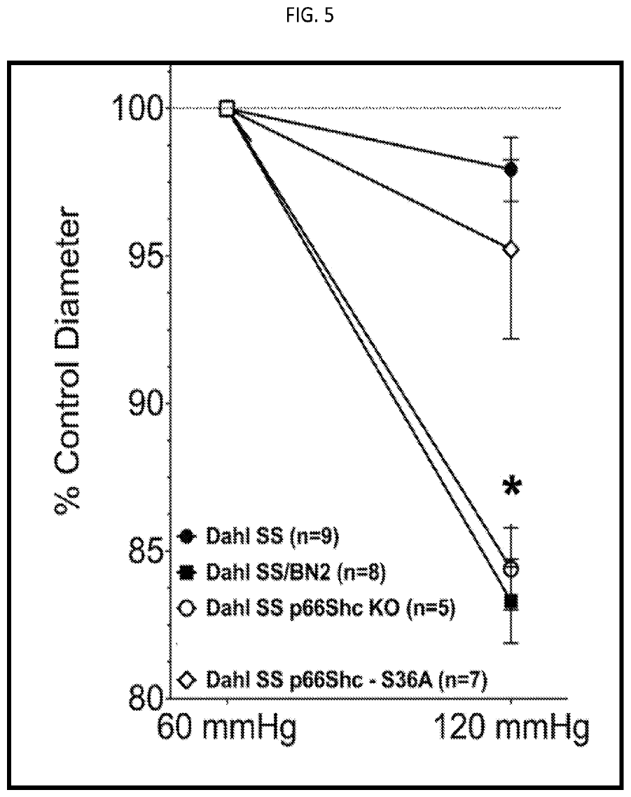

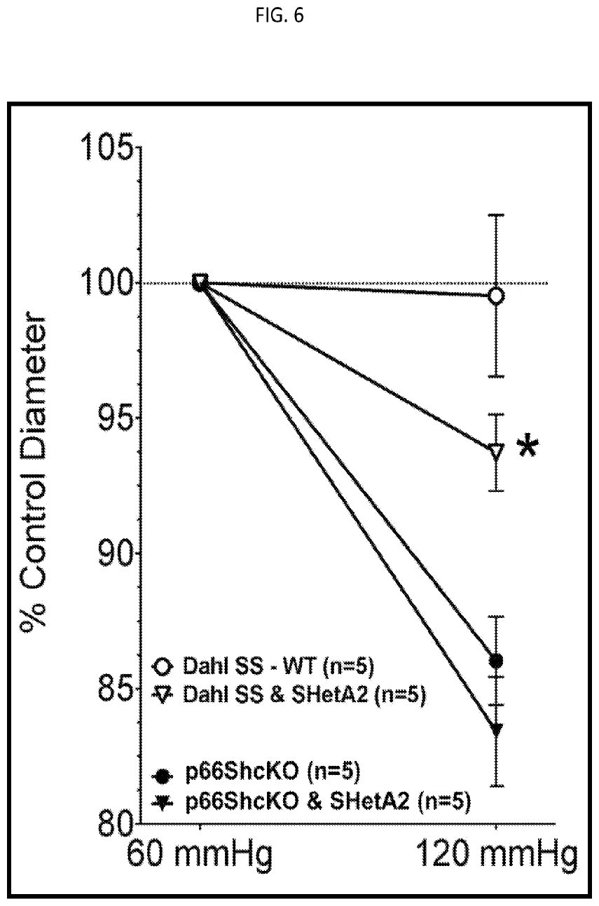

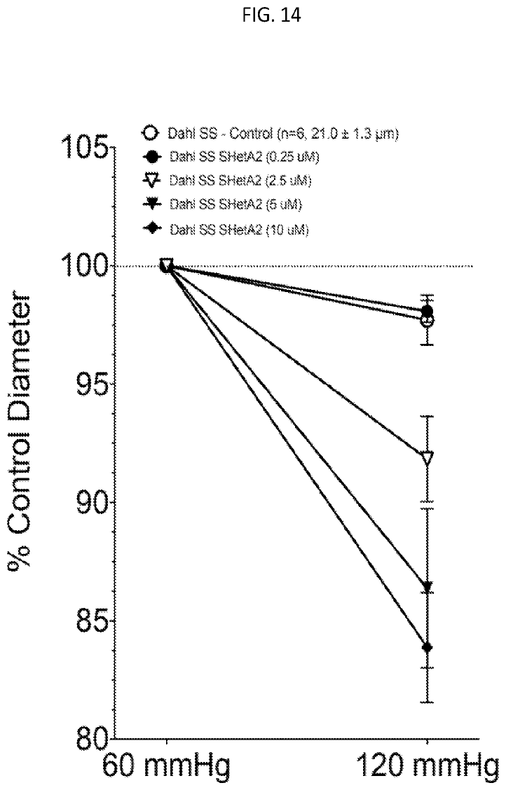

Microvascular reactivity of renal blood vessels is reduced in two major causes of end stage renal disease: hypertension-induced nephropathy (HN) and diabetic nephropathy. One of the highlights of vascular dysfunction in HN is the loss of autoregulation of blood resistance vessels. Using rat model of salt-sensitive (SS) HN we have previously shown that overexpression of adaptor protein p66Shc is implicated in the loss of microvascular reactivity during the progression of HN (FIGS. 3, 5). In HN p66Shc is over-expressed in vascular smooth muscle cells of the walls of renal resistance arteries, including afferent arteriole (FIG. 4). p66Shc knockout restores the ability of afferent arteriole to contract in response to increased perfusion pressure and ATP (FIG. 5) and mitigates glomerular damage in SS rats on 1% salt diet. In this study, we tested ability of a flexible heteroarotinoid called Sulfur Heteroarotinoid A2 (SHetA2, pronounced Ess-Het-Aye-Two), known to inhibit p66Shc action, to prevent development and restore renal microvascular responses in hypertensive rats when added after the development of hypertension-induced nephropathy. Moreover, SHetA2 causes a dose-dependent increase in renal microvascular response in salt-sensitive rats with hypertension (FIG. 14). Remarkably, SHetA2 prevented loss of microvascular reactivity and restored renal microvascular responses in hypertensive rats, as detected by response of afferent arterioles either to increasing perfusion pressure or to ATP, known mediator of afferent arteriolar autoregulatory responses (FIG. 6).

Thus, the small molecule drug SHetA2 mitigates renal damage, prevents loss of microvascular reactivity and restores microvascular responses in hypertensive rats. Since compound SHetA2 demonstrates a reasonable pharmacokinetics and lack of mutagenicity, carcinogenicity and toxicity, it may be beneficially used for therapeutic intervention of p66Shc-mediated vascular diseases.

### Example 2

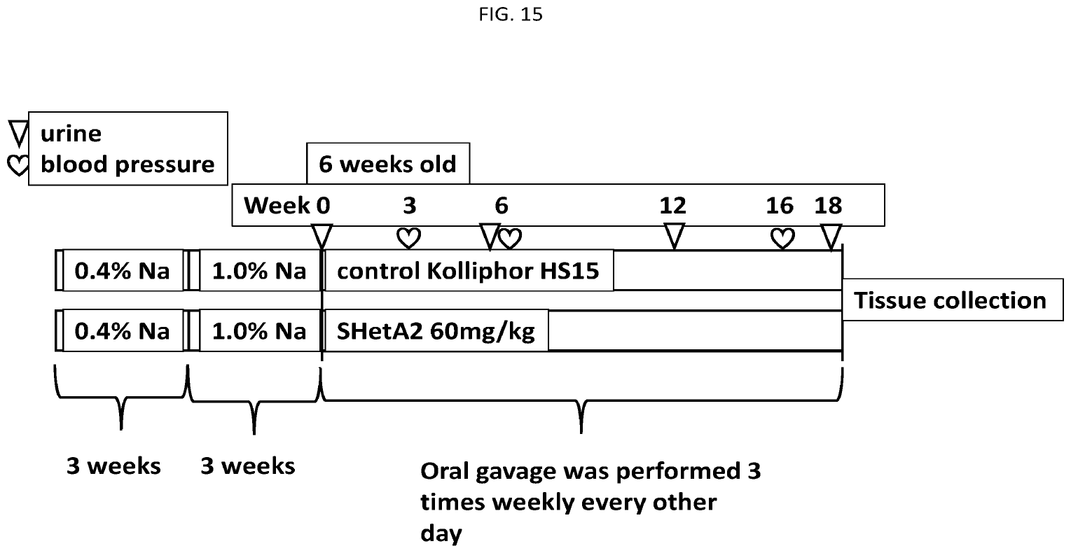

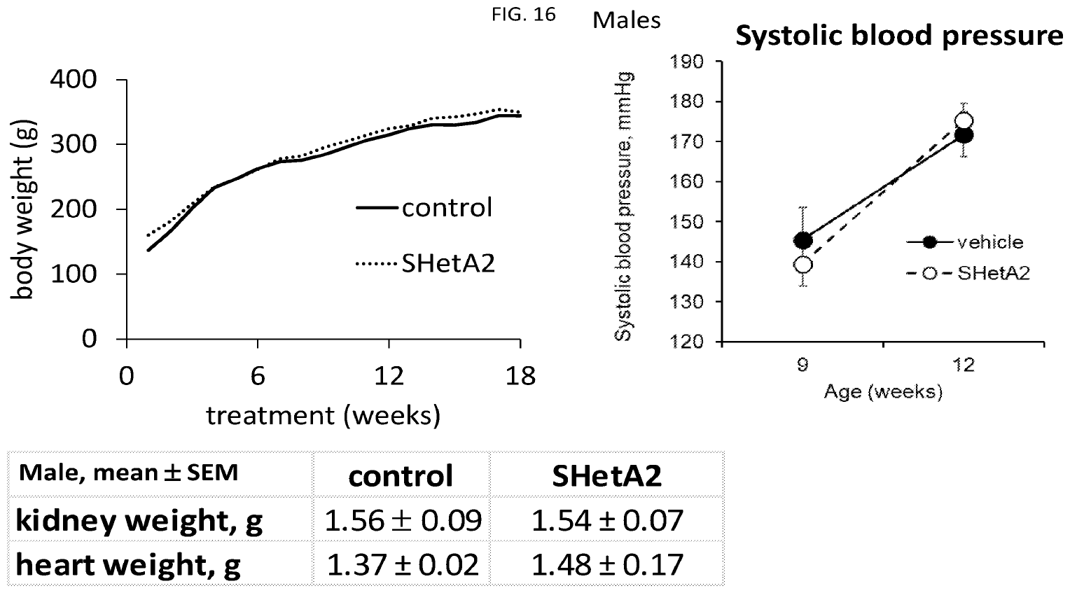

The inventors demonstrated that SHetA2 prevents development of microvascular reactivity and restores renal microvascular responses in a rat model of hypertension-induced nephropathy (FIG. 15). To assess whether treatment with SHetA2 would reduce the systolic blood pressure in hypertensive rats, animals were treated for 18 weeks with either vehicle control or SHetA2. SHetA2 did not significantly reduce the systolic blood pressure of hypertensive rats (FIG. 16). Interestingly, these data indicate that SHetA2 has action at the source of microvascular damage, rather that reducing hypertension in the animal systemically.

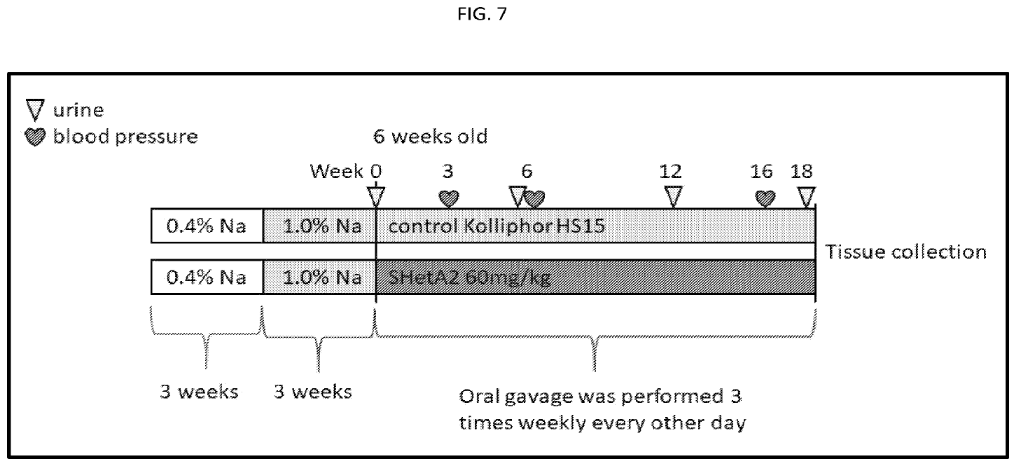

SS rats were weaned at 3 weeks of age and transferred to 1% salt diet for additional 3 weeks before treatment. At 6 weeks of age, rats were either given vehicle control or SHetA2 at 60 mg/kg by oral gavage. Oral gavage was performed 3 times weekly every other day for a period of 18 weeks. Measurements of blood pressure confirmed that SHetA2 treatment did not prevent progression of hypertension caused by 1% salt diet (FIG. 16). Urine was collected from rats housed in metabolic cages for 1-2 hours, approximately 24 hours after last treatment after 6, 12 and 18 weeks of treatment. SHetA2 treatment resulted in statistically significant decrease of urinary protein excretion when compared to control-treated rats, indicating that SHetA2 preserves kidney function in hypertensive rats. Vascular reactivity of renal afferent arterioles was measured using the juxtamedullary nephron technique approximately 24 hours after final treatment (18 weeks) (FIG. 7).

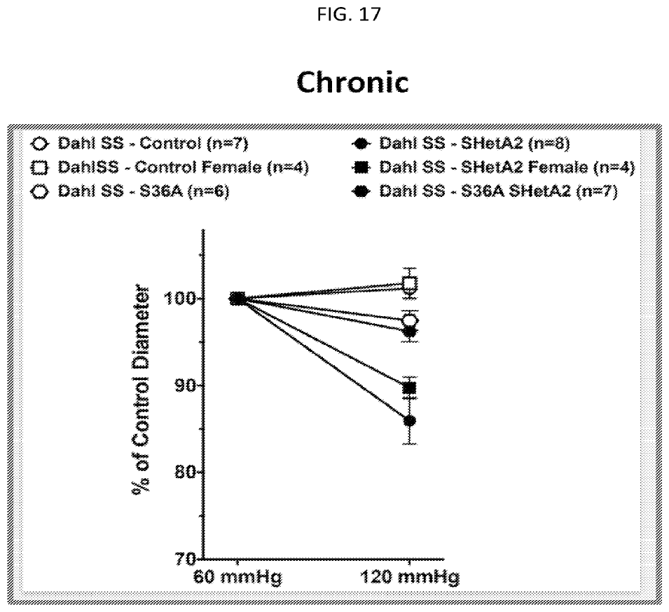

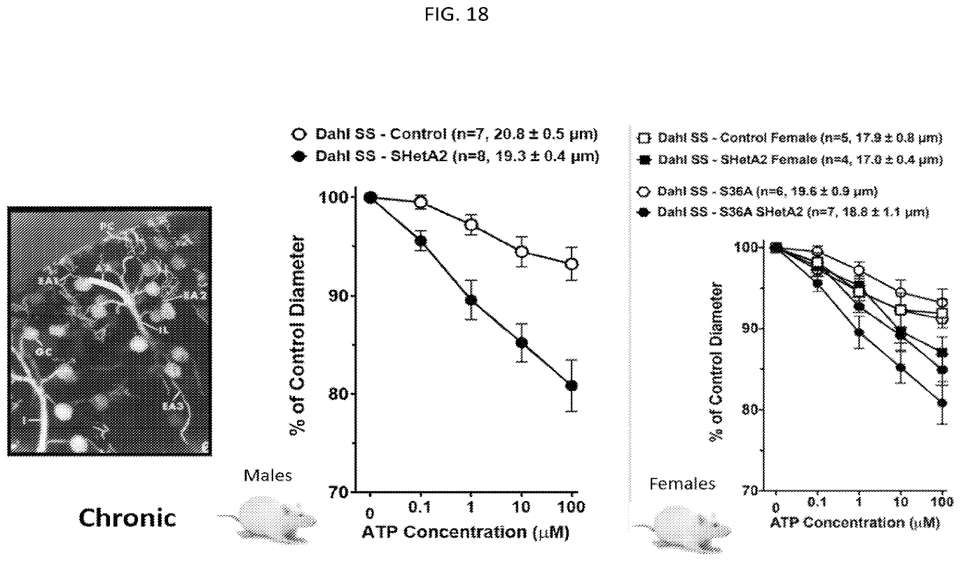

To further elucidate the mechanism by which SHetA2 may be acting to increase renal microvascular responses in hypertensive subjects, the inventors demonstrate that two known inducers of microvascular constriction, pressure (FIG. 17) and addition of exogenous ATP (FIG. 18) stimulate microvascular constriction.

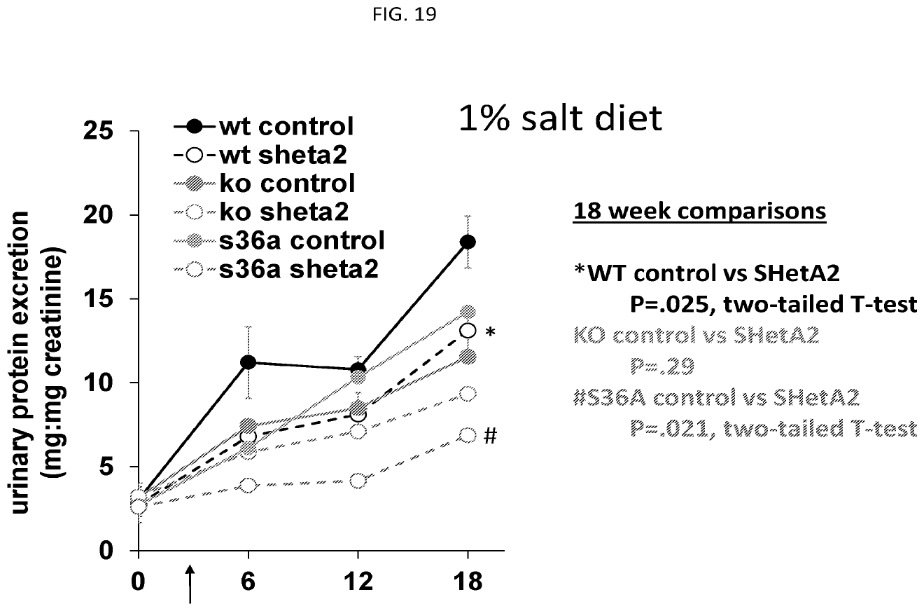

Hypertension-induced nephropathy causes excessive protein excretion in the urine. The inventors demonstrated that treatment of animals with SHetA2 significantly reduces the level of urinary protein excretion in hypertensive animals (FIG. 19, asterisk).

### Diabetic Nephropathy

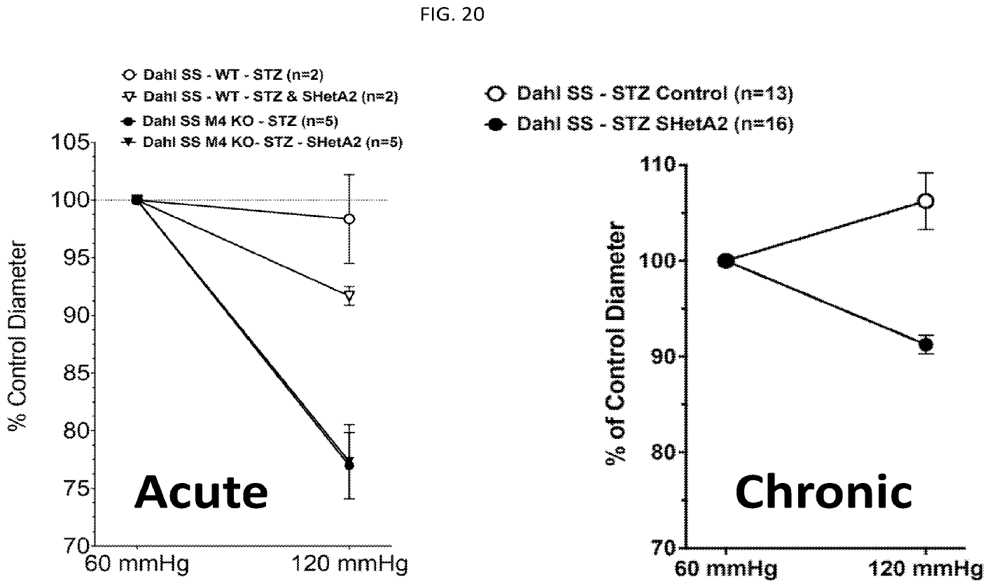

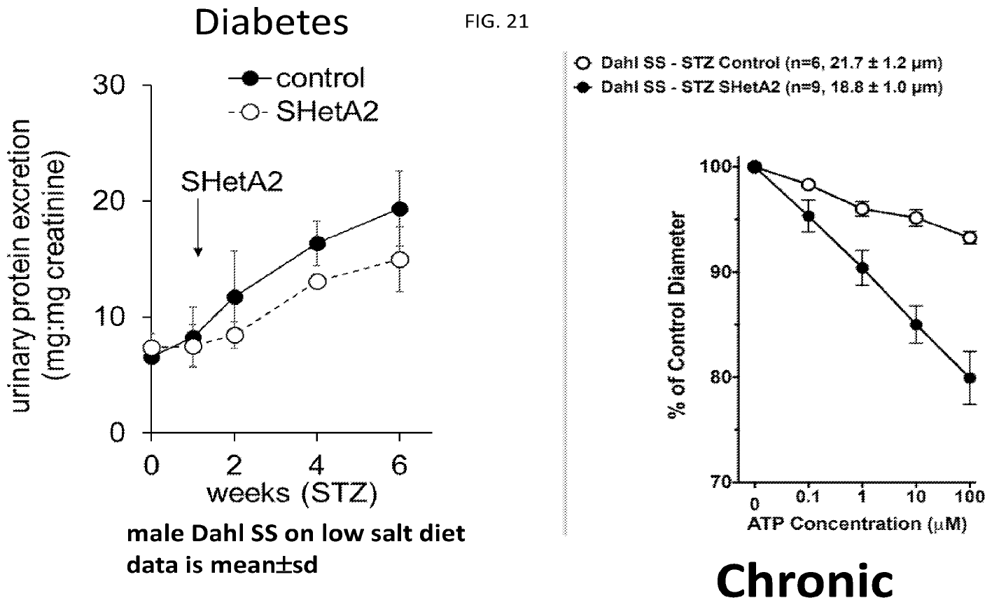

The inventors hypothesized that SHetA2 would be effective in preventing and treating diabetic nephropathy. Therefore, the inventors treated rats with diabetes induced by streptozotocin (STZ) with SHetA2. The inventors demonstrated that SHetA2 successfully prevented development of microvascular damage and increased renal microvascular responses in diabetic animals after acute treatment with SHetA2 and, more important, after chronic treatment of diabetic animals with SHetA2 (administrated by oral gavage every other day). (FIGS. 20-21).

### Mechanism of Action

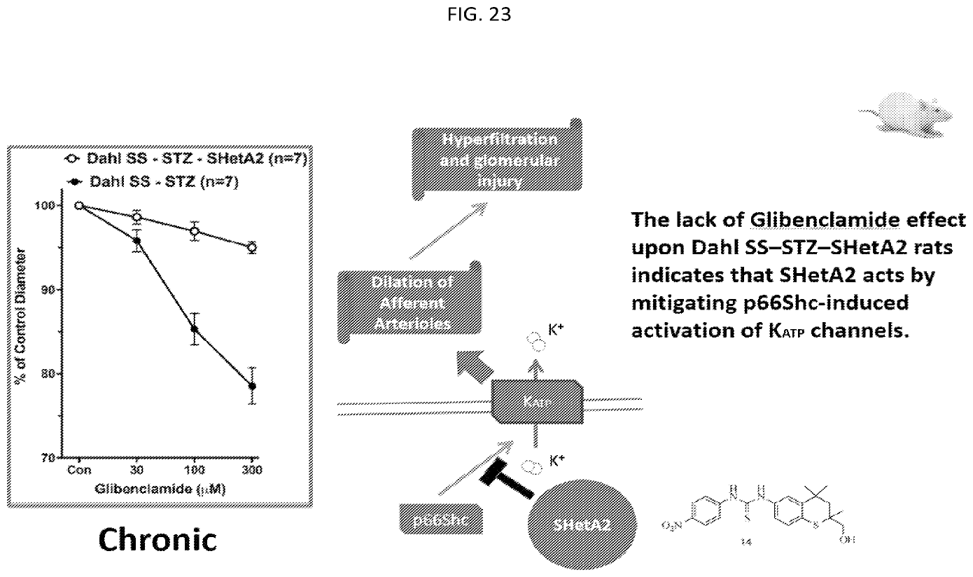

The inventors hypothesized that SHetA2 acts by inhibiting p66Shc-induced activation of K_{ATP} channels in diabetic animals. Therefore, the inventors treated control and SHetA2 treated arterioles with increasing concentrations of glibenclamide, antidiabetic drug which binds and inhibits the ATP-sensitive potassium channel's K_{ATP} inhibitory subunit which causes increasing of intracellular Ca²⁺ concentrations leading to arteriole constriction. The inventors demonstrated that increasing glibenclamide concentrations caused constriction in control arterioles, but not in SHetA2 treated arterioles, supporting the hypothesis that SHetA2 acts by inhibiting p66Shc-induced activation of K_{ATP} channels (FIG. 23) and can prevent development of microvascular damage.

### Conclusion

The inventors conclude that SHetA2 acts to prevent and inhibit p66Shc-induced deleterious effects secondary to hypertension and diabetes, especially with respect to cardiovascular diseases of the kidney vasculature. Thus, SHetA2 is a promising drug for the prevention and treatment of at least two major causes of end-stage renal disease: diabetic nephropathy and hypertension-induced nephropathy, in addition to prevention and treatment of microvascular consequences of other diseases associated with hypertension.

We claim:

1. A method of:
(a) treating a hypertension-linked disease in a subject in need thereof;
(b) delaying the onset of or reducing the incidence of a hypertension-linked disease in a subject in need thereof;
(c) preventing, inhibiting, reducing, or treating p66Shc-mediated vascular diseases in a subject in need thereof;
(d) reducing, inhibiting, or preventing hypertension-induced nephropathy in a subject in need thereof;
(e) preventing, reducing or inhibiting high blood pressure related damage in a subject in need thereof,
the method comprising administering a therapeutically effective amount of SHetA2 or composition comprising SHetA2 to treat the hypertension-linked disease, delay the onset of or reduce the incidence of a hypertension-linked disease, prevent, inhibit, reduce, or treat the p66Shc-mediated vascular disease, prevent, reduce or inhibit the hypertension-induced nephropathy, prevent, or reduce or inhibit high blood pressure related damage.

2. (canceled)

3. The method of claim 1(a) or 1(b), wherein the blood pressure in the subject is not reduced.

4. The method of claim 1(a) or 1(b), wherein the hypertension-linked disease comprises hypertension-induced nephropathy.

5. The method of claim 1(a) or 1(b), wherein the method reduces urinary protein excretion in the subject.

6. The method of claim 1(a) or 1(b), wherein the method prevents dysregulation or improves autoregulation of blood vessels in the subject.

7. (canceled)

8. The method of claim 1(c), wherein the vascular disease is a hypertension-induced disease.

9. The method of claim 1(c) wherein the vascular disease is hypertension-induced nephropathy or diabetic nephropathy.

10. The method of claim 1(c), wherein the method reduces, inhibits or prevents development of increased urinary protein excretion or reduces urinary protein excretion in the subject.

11. The method of claim 1(c), wherein the method reduces, inhibits, or prevents dysregulation of blood vessels or improves autoregulation of blood vessels.

12. (canceled)

13. The method of claim 1(d), wherein the method prevents, reduces or inhibits development of increased urinary protein excretion or reduces urinary protein excretion in the subject.

14. The method of claim 1(d), wherein the method reduces, inhibits or prevents dysregulation, or improves, autoregulation of blood vessels.

15. (canceled)

16. The method of claim 1(e), wherein the method prevents development of increased urinary protein excretion or reduces, urinary protein excretion in the subject.

17. The method of claim 1(e), wherein the method prevents, inhibits or reduces dysregulation, or improves autoregulation, of blood vessels.

18. A kit comprising SHetA2 for prevention or treatment of vascular disease.

19. The kit of claim 18, further comprising instructions to administer SHetA2 for prevention or treatment of diabetic nephropathy or hypertension-induced nephropathy.

20. The kit of claim 18, wherein the kit further comprises an additional drug that reduces blood pressure in a subject.

21. The kit of claim 20, wherein the additional drug comprises a diuretic, an angiotensin converting enzyme (ACE) inhibitor, an angiotensin II receptor blocker, a calcium channel blocker, an alpha blocker, a beta blocker, an alpha-beta blocker, an aldosterone agonist, a renin inhibitor, or a central acting agent.
